(19) United States
(12) Patent Application Publication
(10) Pub. No.: US 2022/0378747 A1
(43) Pub. Date: Dec. 1, 2022

(54) TOPICAL COMPOSITION FOR TREATING SKIN WOUNDS

(71) Applicant: Timothy L. Kelley, Barrington, IL (US)

(72) Inventor: Timothy L. Kelley, Barrington, IL (US)

(21) Appl. No.: 17/333,704

(22) Filed: May 28, 2021

(51) Int. Cl. A61K 31/4166; A61K 33/30; A61K 31/197; A61K 31/047; A61K 31/167; A61K 31/717; A61K 45/06; A61K 31/131; A61K 9/00; A61K 9/06; A61P 17/02

(52) U.S. Cl. CPC A61K 31/4166; A61K 33/30; A61K 31/197; A61K 31/047; A61K 31/167; A61K 31/717; A61K 45/06; A61K 31/131; A61K 9/0014; A61K 9/06; A61P 17/02

(57) ABSTRACT

A composition for treating skin wounds of an individual is disclosed. The composition is an extract that contains Zinc Gluconate, Lysine, Taurine, Potassium Gluconate, Carboxy Methyl Cellulose, Glycerin, Allantoin, water, Hydrogen chloride (HCl), Lidocaine, Niacin, Valine, L-Isoleucine, Proline, L-Arginine, Threonine, Glutamine, Histidine, Serine, Asparagine, Tyrosine, Cysteine, and pentapeptide for example, Enkephalin. The composition could be applied to the affected skin for effectively treating skin wounds, reducing expression lines, and relaxing muscle contractions, thereby reducing skin wrinkles, and smoothening skin. The composition is provided in at least any one or more forms include a gel, a spray, liquid, lotion, and a cream. The composition maintains a pH balance ranges from about 6.5 to 7.5.

### BACKGROUND OF THE INVENTION

### A. Technical Field

The present invention generally relates to a composition for effectively treating skin wounds. More specifically, the present invention relates to a topical composition for effectively treating skin wounds, relaxing muscle contractions, thereby reducing expression lines and skin wrinkles, and smoothening skin.

### B. Description of Related Art

People wish to appear youthful and maintain healthy, vibrant, and a wrinkle-free appearance for as long as possible and consequently seek to prevent, reduce, improve, or eliminate the signs of aging (and/or sun exposure) which reveal themselves in the forms of damage with fine lines, expression lines, furrows, and wrinkles. Expression lines are a distinct type of wrinkle that occurs on the facial skin at an early adult age. They have been related anatomically to the facial expression muscles in the periorbital, glabella, forehead, and perioral areas.

Problematic skin issues have exploded the value of the consumer skincare and wound healing markets over the past 50 years, across a very wide range of age and socio-economic demographics. From pediatric psoriasis, acne, and severe diabetic and age-related ulcerations, these and many other conditions are targeted by various non-pharmaceutical lotions and potions. Even where pharmaceuticals are used to treat underlying viral conditions such as shingles, the distressing associated rashes often require supplementary (or “adjunct”) treatment to heal the skin and relieve side-effects such as pain and irritation. Most skin conditions are chronic due to auto-immune, allergic, or consistent exposure to an irritant requires a long-term therapeutic approach to care.

Currently, a wide variety of compositions have been utilized and promoted in the art for mitigating dermatological conditions of the skin which are frequently associated with the process of aging, expression lines, wrinkles, skin healing and repair, exposure to the sun, or the resulting ultraviolet radiation there from, etc. However, these wide varieties of compositions have negative long-term results and do not fall into the category of safe, natural, and approved for long-term use. Unfortunately, most existing products only treat the outward symptoms and few actually help heal the skin. Further, the existing products could cause problems for the consumers such as redness, dryness, swelling, blisters, stinging and burning feeling, itching, and irritation of the skin.

In light of the above-mentioned problems, it would be highly desirable to provide an improved composition and method for effectively treating skin wounds, relaxing muscle contractions, thereby reducing expression lines and skin wrinkles, and smoothening skin without any side effects such as redness, dryness, swelling, blisters, stinging and burning feeling, itching, and irritation of the skin. Further, there is also a need to provide a composition for effectively promoting wound repair and restoring or maintaining the healthy structure of the skin.

### SUMMARY OF THE INVENTION

The present invention generally discloses a composition for treating skin wounds. Further, the present invention discloses a topical composition for treating skin wounds, relaxing muscle contractions, thereby reducing expression lines and skin wrinkles, and smoothening skin.

In one embodiment, the composition is applied to the affected skin of the individual for treating skin wounds of an individual, relaxing muscle contractions, thereby reducing expression lines and skin wrinkles, and smoothening skin without any side effects such as redness, dryness, swelling, blisters, stinging and burning feeling, itching, and irritation of the skin. In one embodiment, the composition could be applied to the affected area for treating skin wounds.

In one embodiment, the composition could be an extract that contains Zinc Gluconate, Lysine, Taurine, Potassium Gluconate, Carboxy Methyl Cellulose, Glycerin, Allantoin, water, Hydrogen chloride (HCl), Lidocaine, Niacin, Valine, L-Isoleucine, Proline, L-Arginine, Threonine, Glutamine, Histidine, Serine, Asparagine, Tyrosine, Cysteine, and pentapeptide i.e., Enkephalin.

In one embodiment, Zinc Gluconate is present in an amount of about 5.0% by weight of said composition, Lysine in an amount of about 0.1%, Taurine in an amount of about 1.0%, Potassium Gluconate in an amount of about 1.0%, Carboxy Methyl Cellulose in an amount of about 0.5%, Glycerin in an amount of about 10%, Allantoin in an amount of about 1.0%, Lidocaine in an amount of about 3%.

In one embodiment, the composition maintains a pH balance ranges from about 6.5 to 7.5. In one embodiment, the composition could be a solution. In one embodiment, the composition could be provided in at least any one or more forms include, but not limited to, a gel, a spray, liquid, lotion, and a cream. In one embodiment, the composition is a less toxic product and the ingredients used in the composition are safe and effective to treat wounds, reduce expression lines and skin wrinkles, and improve the smoothness of the skin.

Other objects, features, and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The embodiments herein will be better understood from the following detailed description with reference to the drawings, in which:

FIG. 1 exemplarily illustrates a table that represents a list of ingredients used in a topical composition and their respective values by weight of the topical composition in percentage, according to an embodiment of the present invention.

FIG. 2 exemplarily illustrates a flowchart of a method for treating skin wounds using the topical composition, according to one embodiment of the present invention.

FIG. 3 exemplarily illustrates a screenshot of composition's actions and effects on the skin, according to one embodiment of the present invention.

FIG. 4 exemplarily illustrates a screenshot of a trailer (i.e., the composition) delivering to the target cells, according to one embodiment of the present invention.

### DETAILED DESCRIPTION OF EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive.

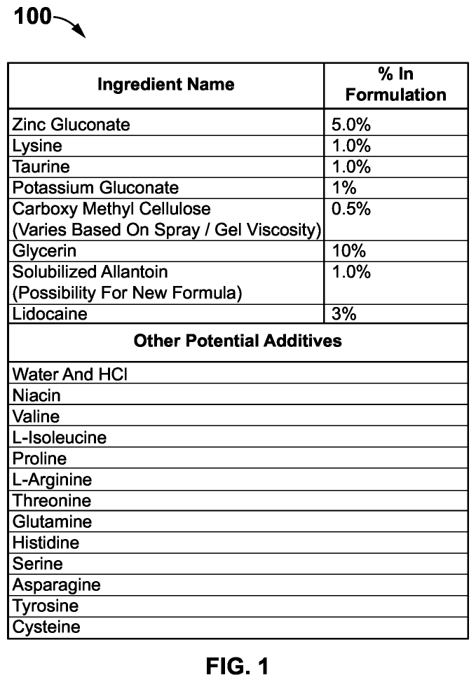

Referring to FIG. 1, a table 100 represents a list of ingredients used in a topical composition and their respective values by weight of the composition in percentage in one embodiment is disclosed. In one embodiment, the composition could be used for treating skin wounds of an individual, relaxing muscle contractions, thereby reducing expression lines and skin wrinkles, and smoothening skin without any side effects such as redness, dryness, swelling, blisters, stinging, and burning feeling, itching, and irritation of the skin. In one embodiment, the composition could be applied to the affected area for treating skin wounds.

In one embodiment, the composition could be an extract that includes Zinc Gluconate, Lysine, Taurine, Potassium Gluconate, Carboxy Methyl Cellulose, Glycerin, Allantoin, water, Hydrogen chloride (HCl), Lidocaine, Niacin, Valine, L-Isoleucine, Proline, L-Arginine, Threonine, Glutamine, Histidine, Serine, Asparagine, Tyrosine, Cysteine, and pentapeptide i.e., Enkephalin.

In one embodiment, Zinc Gluconate is present in an amount of about 5.0% by weight of said composition, Lysine in an amount of about 0.1%, Taurine in an amount of about 1.0%, Potassium Gluconate in an amount of about 1.0%, Carboxy Methyl Cellulose in an amount of about 0.5%, Glycerin in an amount of about 10%, Allantoin in an amount of about 1.0%, Lidocaine in an amount of about 3%.

In one embodiment, the composition maintains a pH balance ranges from about 6.5 to 7.5. In one embodiment, the composition could be a solution. In one embodiment, the composition could be provided in at least any one or more forms include, but not limited to, a gel, a spray, liquid, lotion, and a cream.

In one embodiment, Zinc gluconate contains at least 14.3% of elemental zinc, which is essential for healing skin wounds. Zinc is a limiting factor in the formation of RNA and DNA. In one embodiment, the composition works by presenting zinc in a bio-available form to target cells involved in wound healing. Zinc is involved in certain enzymes essential for the maintenance of life in man, animals, and plants. In some instances, vitamins facilitate the incorporation of the mineral into the enzyme such that enzyme activity is inhibited by a shortage in the mineral or the vitamin. For example, zinc is involved in the synthesis of DNA by the zinc-containing enzyme DNA polymerase. The vitamin niacin facilitates the incorporation of zinc into the peptide subunits of the DNA polymerase enzyme. If either niacin or zinc is deficient in the body, DNA polymerase activity of the tissues may be reduced and the result in both cases is lack of growth (and therefore retardation of healing).

In one embodiment, the composition applied to the skin surface may permeate through the skin and penetrate into the skin layers or the target tissue in the correct concentration for a sufficient length of time, thereby achieving the desired therapeutic effect. The composition could be absorbed through the skin of the individual. The composition is developed for the administration of zinc in a bio-available form, which is readily absorbed into the serum which then passes into the target cells.

In one embodiment, Lysine is a basic amino acid (positively charged at pH 6) and might be used to neutralize the acidity of zinc salts such as zinc acetate, zinc chloride, and zinc sulfate. In one embodiment, zinc gluconate unlike zinc acetate, zinc chloride, and zinc sulfate, could be neutralized with the basic amino acids to provide a stable mixture. In one embodiment, zinc gluconate could be neutralized with a base such as sodium hydroxide in the presence of certain non-polar amino acids and certain uncharged polar amino acids to provide a water-soluble stable mixture. Zinc present in the composition could be absorbed more readily and acts more efficiently on the cellular level in the skin tissue.

In one embodiment, the composition mainly includes Zinc gluconate, Lysine, and Taurine. In one embodiment, Zinc gluconate is a zinc salt of gluconic acid, which contains zinc needed for healing skin. It is essential to understand that zinc gluconate in and of itself is not readily absorbed by the skin. In one embodiment, Lysine is an amino acid, which is added to neutralize zinc gluconate in the composition to create the absorbable compound. Lysine is commonly used in the manufacture of medicine as a delivery vehicle to human cells and represents the differentiating factor in the composition formulation. Lysine is a natural amino acid of choice for the composition because of its properties and could be applied directly to the skin (topically). Lysine may treat the herpes simplex conditions such as cold sores and fever blisters and may also reduce healing time. In one embodiment, Taurine is an organic compound that is widely distributed in animal tissues. It is used for its anti-fibrotic properties, which help prevent scarring, along with its usefulness in hydrating the skin and protecting hair follicles.

In another embodiment, the composition contains Lidocaine in the amount of about, but not limited to, 0.5% by weight of said composition. In one embodiment, the composition contains 0.5% of Lidocaine is comfortably used on the skin to stop itching and pain caused by sunburn, minor burns, insect bites, stings, poison ivy, oak, and sumac, minor cuts, scratches, scrapes, and eczema. Lidocaine is considered the safest topical anesthetic available.

In yet another embodiment, the composition contains Cannabidiol (CBD) oil in the amount of about, but not limited to, 1% by weight of said composition. Cannabidiol is a popular natural remedy used for many common ailments. CBD oil could be an appealing option for those who are looking for relief from pain and other symptoms without the mind-altering effects of marijuana or certain pharmaceutical drugs. The CBD oil is made by extracting CBD from the Cannabis plant, then diluting it with a carrier oil like coconut or hemp seed oil. The CBD oil may ease symptoms of ailments like chronic pain and anxiety. The CBD may help reduce chronic pain by impacting the endocannabinoid receptor activity in the consumer, reducing inflammation, and interacting with neurotransmitters.

In one embodiment, the composition could be, but not limited to, a solution. In one embodiment, the composition could be stored and packaged in a bottle or a tube in the form of a gel or a solution. In one embodiment, the composition is a less toxic product and the ingredients used in the composition are safe and effective to treat wounds, reduce expression lines and skin wrinkles, and improve the smoothness of the skin.

In one embodiment, the composition in the form of a gel is slightly thicker than the spray and is primarily used for highly localized cuts and abrasions and particularly for dressed deep tissue ulcers where longer in-dwelling of the product. In one embodiment, the composition in the form of a spray could be used to effectively treat larger areas, which are more sensitive to touch such as generalized rashes, for example, diaper rash, shingles, psoriasis, and burns, etc. In one embodiment, the composition in the form of a spray could also particularly useful in cases where infection control is a concern such as generalized burns where infection risk is high.

In one embodiment, the composition further comprises pentapeptide, which is effective in reducing expression lines as an alternative to a botulinum toxin type mechanism. Enkephalin is a pentapeptide involved in regulating nociception in the body. Enkephalin inhibits the flow of calcium ions into neurons, reduces excitation, prevents the fusion of synaptic vesicles with the presynaptic membrane, and releases acetylcholine. It has a synergistic effect with Argireline and is used against expression lines. It also enhances the effect of relaxing the facial muscles, reduces muscle contractions, and protects the skin from the damaging effects of free radicals. It could effectively reduce the depth of facial wrinkles, especially in the forehead and around the eyes.

In one embodiment, the composition could effectively reduce the depth of facial wrinkles caused by the contraction of facial muscles, especially on the forehead and around the eyes. In one embodiment, the composition acts on the mechanism of the formation of expression lines in a new way, thereby offering an alternative to other peptides (like Argireline) and effectively softens the skin. In one embodiment, the composition relaxes muscle contractions, thereby preventing skin wrinkles.

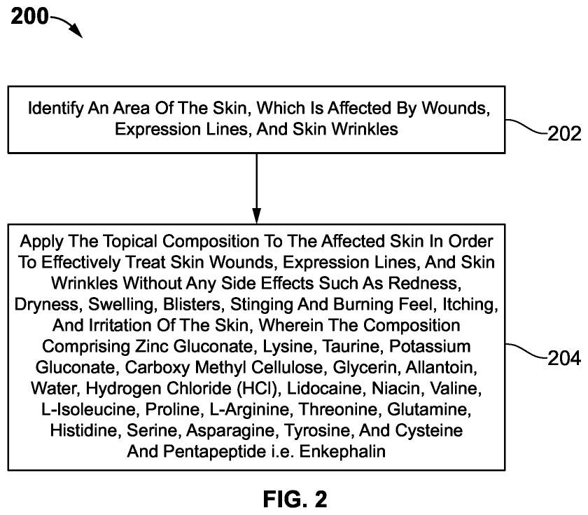

Referring to FIG. 2, a flowchart 200 of a method for treating the skin wounds using the topical composition in one embodiment is disclosed. At step 202, the user or consumer could identify an area of the skin, which is affected by wounds, expression lines, and skin wrinkles. Further, at step 204, the user could apply the topical composition to the affected skin in order to effectively treat skin wounds, expression lines, and skin wrinkles without any side effects such as redness, dryness, swelling, blisters, stinging and burning feeling, itching, and irritation of the skin.

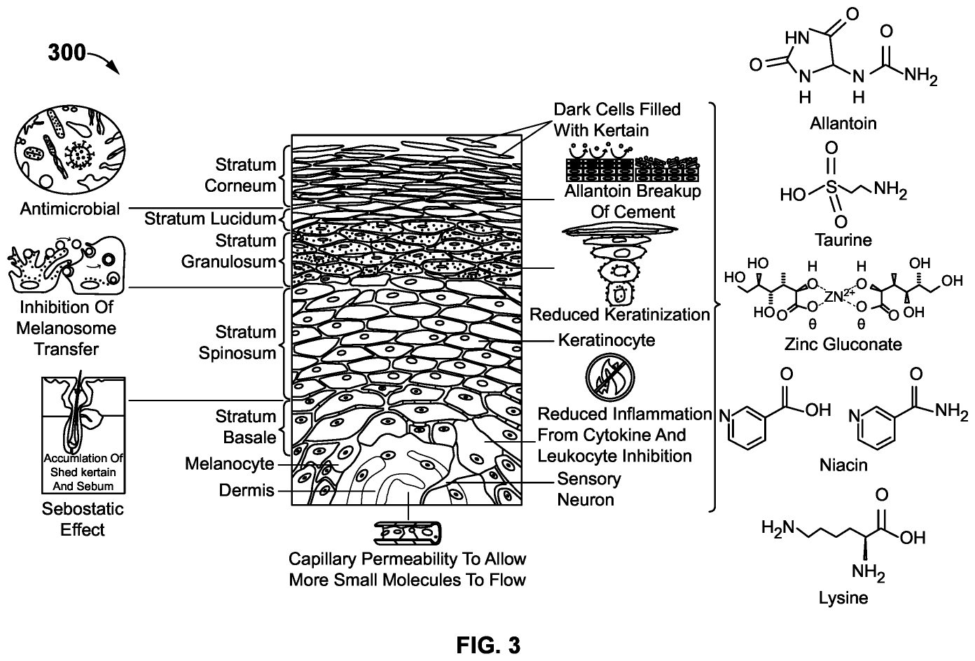

Referring to FIG. 3, a screenshot 300 of composition's actions and effects on the skin in one embodiment is disclosed. The composition of the present invention could effectively work on the skin and advantageously obtain results by delivering bio-available zinc to the target cells. The composition could provide additional therapeutic value through enhanced epithelization, anti-bacterial action, anti-viral action, and accelerated healing action of the skin.

Allantoin present in the composition is a mild keratolytic agent that dissolves the intercellular cement, which holds the cornified cells together, helps the natural desquamation of stratum corneum and increases epidermal smoothness. Allantoin has multiple properties/effects to facilitate transition of a wound from an inflammatory to a proliferative state, including antioxidant and anti-inflammatory properties, direct antimicrobial effects, and keratolytic activity facilitating wound healing. The composition could reduce inflammation from cytokine and leukocyte inhibition. Allantoin has been shown to facilitate proliferation of healthy tissue by promoting cell proliferation and extracellular matrix synthesis. Allantoin may also have a role in tissue formation and differentiation, specifically in stimulating the development of granulation tissue and epithelialization. It may also reduce scar formation by preventing epidural fibrosis, as tested in a rat hemilaminectomy model. It is a derivative of urea exhibits keratolytic action by affecting desmosomy, which are connections between corneocytes. Allantoin by doing so you get rid of excess keratinized epidermis. At the same time by stimulating cell division and epithelization, accelerates the regeneration process of damaged skin inflammation. It also has strong moisturizing properties and is not allergenic, and does not cause skin irritation. Because of these properties, allantoin is widely used in cosmetics and pharmacy.

Allantoin contained in an ointment or cream accelerates the exfoliation of dead skin cells, making the skin smoother. Further accelerating the proliferation of epithelial cells also occurs to quicker recovery of the skin, making skin more easily retain more water and to rebuild the protective hydro-lipid coat. The composition containing allantoin is also recommended as an adjunct in the treatment of chronic skin diseases with impaired keratosis or skin damage, such as atopic dermatitis, contact dermatitis, psoriasis, ichthyosis, ulcers or burns. Complementary therapy, external preparations containing allantoin, significantly reduces symptoms and improves the quality of life of the patient.

Bioavailable niacin (C₆H₅NO₂) in the form of Niacinimide has been added to the composition to add additional modes of action to the bioavailability of nutrients and capability of delivery to targeted cells. As vitamin and essential nutrient as a catabolistic agent of fat, carbohydrates and proteins, it signals DNA polymerase repair. Niacinamide, an amide form of vitamin B3 (niacin), is a hydrophilic endogenous substance. Given a sufficient bioavailability, niacinamide has antipruritic, antimicrobial, vasoactive, photoprotective, sebostatic and lightening effects depending on its concentration. Within a complex metabolic system niacinamide controls the NFκB-mediated transcription of signaling molecules by inhibiting the nuclear poly (ADP-ribose) polymerase-1 (PARP-1). Niacinamide is a well-tolerated and safe substance often used in cosmetics. Clinical data for its therapeutic use in various dermatoses can increasingly be found in the literature. Niacin acts as an anti-inflammatory, skin lightning, anti-microbial, barrier protection, and photoprotective.

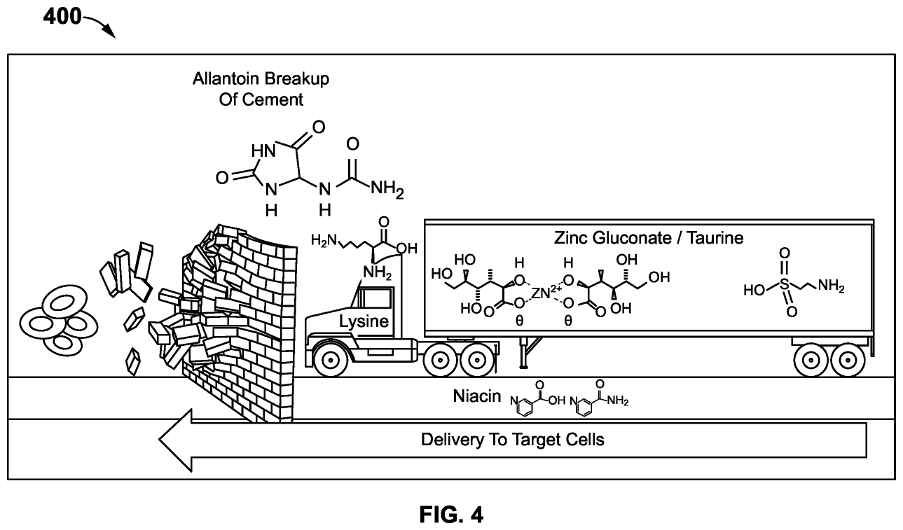

Referring to FIG. 4, a screenshot 400 of a trailer (i.e., the composition) delivering to the target cells in an exemplary embodiment is disclosed. For example, a fully loaded Walmart trailer (i.e., the composition) without a tractor directed to the target cells. Hitch the tractor to the trailer, and the delivery is made. Zinc in the composition could be safely driven deep into the user's tissue where it is needed by hitching the all natural tractor and trailer ingredients in the composition (zinc gluconate and amino acids) to help clear up psoriasis, eczema, diaper rash, and even difficult and persistent wounds.

In accordance with the present invention, a mineral salt of a carboxylic acid derivative of a pentose or hexose such as zinc gluconate or zinc gluconate is neutralized in the presence of certain amino acids to provide minerals in bioavailable form which are absorbed more readily and act more efficiently on the cellular level in the target organ than minerals alone. Other minerals which may be administered in the subject form include calcium, iron, magnesium, manganese and the like. Zinc gluconate, like zinc acetate, zinc chloride and zinc sulfate, is acidic as illustrated by the following table:

| | pH of Zinc Solutions | |
|---|---|---|
| | pH | |
| Solutions | 0.1M 0.2M | |
| Zinc acetate | 6.7 | 6.6 |
| Zinc chloride | 6.4 | 6.2 |
| Zinc sulfate | 5.7 | 5.45 |
| Zinc gluconate | 5.85 | 5.65 |

In common with the other soluble zinc salts mentioned above, zinc gluconate cannot be neutralized with sodium hydroxide without precipitating the mixture. It has been discovered, however, that the acidity of the zinc gluconate can be neutralized by adding the following amino acids and adjusting the pH to 7.0: alanine, valine, isoleucine, proline, glycine, serine, threonine, asparagine, glutamine, lysine, arginine, histidine and mixtures thereof. The adjustment cannot be made with cysteine, tyrosine, aspartic acid or glutamic acid.

In general, it is preferred that the mineral salt and the amino acid be present in substantially equimolar amounts. Suitable therapeutic formulations are formed with a molar amount of mineral salt such as zinc gluconate to amino acid from about 0.05M:1.0M to about 1.0M:0.05M, preferably from about 0.1M: 0.3M to 3M:0.1M and most preferably from about 0.1M:0.1M to 0.3M:0.3M and neutralized to a pH in the range from about 6.0 to 8.0, preferably from about 6.5 to 7.5 and most preferably 7.0.

As discussed below, the amino acids suitable for use in accordance with the present invention cut across the classification groups. There are eight standard amino acids having nonpolar R groups. Five have aliphatic hydrocarbon R groups (alanine, leucine, isoleucine, valine and proline), two with aromatic rings (phenylalanine and tryptophan) and one containing sulfur (methionine). Of these, as shown in the following table, alanine, valine, isoleucine and proline are suitable for use as described herein, whereas leucine, phenylalanine, tryptophan and methionine are not:

0.1M Zinc Gluconate and 0.1M Amino Acid Solutions

| Nonpolar Amino Acids | pH | Observations |
|---|---|---|
| Alanine | 5.1 | pH adjusted to 7.0 with NaOH does not |
| | | precipitate |
| Valine | 5.1 | pH adjusted to 7.0 with NaOH does not |
| | | precipitate |
| Leucine | 5.0 | pH adjusted to 7.0 with NaOH precipitates |
| Isoleucine | 5.1 | pH adjusted to 7.0 with NaOH does not |
| | | precipitate |
| Proline | 5.4 | pH adjusted to 7.0 with NaOH does not |
| | | precipitate |
| Phenylalanine | 4.9 | pH adjusted to 7.0 with NaOH |
| | | precipitates |
| Tryptophan | | Not soluble |
| Methionine | 5-1 | pH adjusted to 7.0 with NaOH precipitates |

There are seven standard amino acids with uncharged polar R groups which can hydrogen-bond with water. The polarity of serine, threonine and tyrosine is contributed by their hydroxyl groups; that of asparagine and glutamine by their amide groups and that of cysteine by its sulfhydryl group. Glycine is sometimes classified as a nonpolar amino acid but its R group, a single hydrogen atom, is too small to influence the high degree of polarity of the amino and carboxyl groups. Asparagine and glutamine are the amides of aspartic acid and glutamic acid and are easily hydrolyzed by acid or base to aspartic acid and glutamic acid, respectively. Cysteine and tyrosine have the most polar substituents of this class of amino acids, namely the thiol and phenolic hydroxyl groups, respectively. These groups tend to lose protons by ionization far more readily than the R groups of other amino acids of this class, although they are only slightly ionized at pH 7.0. Of the above-mentioned amino acids with uncharged polar R groups, as shown in the following table, glycine, serine, threonine, asparagine and glutamine are suitable for use herein, whereas cysteine and tyrosine are not:

0.1M Zinc Gluconate and 0.1M Amino Acid Solutions

| Amino Acids with | | |
|---|---|---|
| Uncharged Polar R Groups | pH | Observations |
| Glycine | 5.0 | pH adjusted to 7.0 with NaOH does |
| | | not precipitate |
| Serine | 4.8 | pH adjusted to 7.0 with NaOH |
| | | does not precipitate |
| Threonine | 5.0* | pH adjusted to 7.0 with NaOH |
| | | does not precipitate |
| Cysteine | 4:3 | pH adjusted to 7.0 with NaOH |
| | | precipitates |
| Tyrosine | | Not soluble |
| Asparagine | 4.8 | pH adjusted to 7.0 with NaOH does |
| | | not precipitate |
| Glutamine | 5.0 | pH adjusted to 7.0 with NaOH does |
| | | not precipitate |

The basic amino acids, in which the R groups have a net positive charge at pH 7.0, all have six carbon atoms. They consist of lysine, which bears a positively charged amino group at the e position on its aliphatic chain; arginine, which bears the positively charged guanidinium group and histidine, which contains the weakly basic imidazolium function. Histidine is the only amino acid having buffering capacity near pH 7.0 with a pK′ of the R group near 7.0. All of the basic amino acids as shown in the following table are suitable for use in the present invention:

| Amino Acids with (−) | | |
|---|---|---|
| Charged Polar Groups | pH | Observations |
| Lysine | 7.4 | pH adjusted to 7.0 with HC1 does not |
| | | precipitate |
| Arginine | 7.4 | pH adiusted to 7.0 with HC1 does not |
| | | precipitate |
| Histidine | .5.5 | pH adjusted to 7.0 with NaOH does not |
| | | precipitate |

The two amino Acids with negatively charged (acidic) R groups are aspartic acid and glutamic acid, each with a second carboxyl group which is fully ionized and negatively charged at pH 6 to 7. Neither of these amino acids is suitable for use as herein described
as shown in the following table:

0.1M Zinc Gluconate and 0.1M Amino Acid Solutions

| Amino Acids with (+) Charged | |
|---|---|
| Polar Groups | pH Observations |
| Aspartic acid | Not soluble |
| Glutamic acid | Not soluble |

In addition to the standard 20 amino acids commonly found in proteins, there are also synthetic and other naturally occurring amino acids known to occur biologically in free or combined form. Among these other amino acids, other suitable amino acids for use herein may be identified if screened as described above.

In some instances, it is advantageous to administer the neutralized mineral salt in combination with a vitamin such as vitamin C or niacin (b3) and so forth depending on the condition to be treated. Minerals in accordance with the present invention, optionally in combination with other active therapeutic ingredients such as vitamins and other drugs, are utilized in aqueous, emulsion or dried form in combination with other materials such as glycerin, carboxy methyl cellulose, oils, sweeteners, flavors and so forth as carriers or excipients. Depending on the form and the target tissue to be treated, they are taken orally, applied topically to the external or internal epithelium or injected.

### Example 1

The composition is used for treating twenty-eight newborn children, ranging from 3 months to 11 months of age, with diaper rash, redness and irritation were sprayed twice daily for three days. Improvement was seen in all cases and fifty percent of the patients showed improvement by the second day. The composition formula by weight is 1.0% Zinc gluconate 0.3% Lysine 10.0% Glycerin, 0.3% Lysine 10.0% Glycerin, q.s. distilled water and HCL to adjust pH to 7.0.

### Example 2

The composition is used for treating wounds. Thirty-six pigs in confinement on a farm had wet wounds. Some of the pigs had wounds on the ears, due to biting and fighting, and some had wounds on the ears due to scratches with wire. The wounds had been previously treated with a sulfa drug for wound healing but the wound did not heal and continued to exist. The wounds were sprayed twice daily for two days with the following formula. The composition formula by weight is 5.0% Zinc gluconate 1.5% Lysine 10.0% Glycerin, q.s. distilled water and HCl to adjust pH to 7.0. Within two days, the ulceration began to heal, the bleeding stopped, and the condition improved. The ulceration was no longer visible after two weeks, and the skin returned to normal within one month.

### Example 3

The composition is used for treating irritated skin and redness. The irritated skin of the thighs and buttocks of one 6-month old baby and three children, aged 2 to 21 years, were treated with the composition. The composition formula by weight is 1.0% Zinc gluconate, 0.3% Lysine, q.s. Lotion consisting of an emulsified hydrogenated mixture of olive, palm and coconut oils and HCl to adjust pH to 7.0. The irritation, redness and completely vanished in five days.

### Example 4

The composition is used for treating patients with cornification of the heel and dryness of the feet. Seven diabetic patients with cornification of the heel and dryness of the feet were sprayed with the above formula as that used for treating diaper rash in Example. The lotion was applied overnight and this regimen was followed for one week. After one week, the dryness, scaling and odor of the feet had vanished and the skin was softened.

### Example 5

The composition is used for decreasing both odor and bacteria in shoes. Seventeen patients were asked to spray the inside of their shoes with a formulation consisting of 5.0% zinc gluconate, 1.5% lysine percent by weight and q.s. water and HCl to adjust pH to 7.0. Swabs taken before the shoes were sprayed evidenced heavy growth of bacteria while swabs taken 8 hours later evidenced scant growth. The spray decreased both odor and bacteria in the shoes.

### Example 6

The composition is used for treating dogs with hot spot, scales, dry skin, hair loss and pathological documentation of tinea vulgaris on the skin. A gel is applied twice daily for five days to three dogs. The composition percent by weight is 2.0% Carboxy methyl cellulose, 5.0% Zinc gluconate 1.5% Lysine, 0.05% Mandelic Acid, q.s Water and HCl to adjust pH to 7.0. After five days, the redness and scales had vanished and, within two weeks, hair growth had resumed. One of the dogs had Demodex mites under the skin and was treated with the gel for ten days. The gel inhibited the growth of the mites and the hair resumed growth.

### Example 7

The composition is used for treating dogs experienced recurrence of chronic dermatitis. Four dogs experienced recurrence of chronic dermatitis during the course of six to 18 months despite treatment with different antibiotics and cortisones. Dermatitis in dogs occurs as a result of allergic reaction which is not fully understood. The following formulation was added once daily to the drinking water of each dog on the basis of one ml of the solution per one kilogram of body weight. The composition percent by weight is 1.0% Zinc gluconate, 5.0% Potassium gluconate, 0.5% Lysine, 2.0% Taurine, and q.s. Beef flavored water and HCl to adjust pH to 7.0. Each ml of the solution contained one mg of zinc gluconate and 4 mg of potassium gluconate, 0.5 mg of lysine and 2 mg of taurine. After two weeks of this regimen, the dry and scaly skin of all four dogs showed improvement and the hair coat appeared healthier. By the end of one month, there was no evidence of dermatitis whatsoever. During the three-month follow-up period to date, the dermatitis has not recurred although episodes of dermatitis generally increase during hot and dry weather characteristic of the last two months of the follow-up period. None of the dogs exhibited signs of toxicity, stomach irritation, diarrhea or vomiting due to ingestion of zinc gluconate at the recommended dose based on body weight.

### Example 8

The composition is used for treating dogs with dryness and inflammation of the feet/—paws. During the months July and August, characterized by drought conditions and extreme heat, many dogs suffer dryness and inflammation of the feet/—paws. The following was formulated as a foot ointment or spray for prevention of burning and drying of the feet/paws caused by the extreme conditions: The ointment was applied to the feet/paws once daily for two months. The dogs were examined daily for signs of discomfort. The condition improved and protection was achieved by the end of the two-month treatment period. The composition of the ointment percent by weight is 2.0% Carboxy methyl cellulose, 5.0% Zinc gluconate, 1.5% Lysine, 1.5% Histidine, and q.s. Water and HCl to adjust pH to 7.0.

### Example 9

The composition is used for treating dogs with halitosis and fungus growth on the tongue. On a daily basis for two weeks, six dogs with halitosis, one of which also had fungus growth on the tongue, were fed two dog biscuits (20 grams per day) that had been impregnated with the composition. The composition percent by weight is 5.1% Zinc gluconate, 1.55% Histidine, 1.74% Arginine, 1.46% Lysine, and q.s. Water and HCl to adjust pH to 7.0. After two weeks, the halitosis had disappeared and the hygienic conditions of the oral cavity improved. The dark spots on the tongue of the one dog that had fungus growth were no longer visible and the tongue resumed its normal appearance.

### Example 10

Thirty-five g of ascorbic acid (2M) is dissolved in 150 ml water and cooled to 4 C. The initial pH of the solution was 2. Eight g of zinc oxide (1M) was added while the solution was continuously stirred and kept cool. After a few hours, all of the zinc oxide went into solution and pH rose to 4.9. The mixture which was light yellow in color was immediately frozen and dried under vacuum. This yielded a very light-yellow colored powder with a molecular weight of 415.61.

### Example 11

A formulation in powder form containing zinc and lysine which when dissolved in water forms a neutral solution was prepared as follows: Two M lysine and 1M zinc gluconate was dissolved in water to form a solution having a pH 7.4 and was neutralized to pH 7 with HCl. After the zinc and amino solution was neutralized, it was frozen immediately in liquid nitrogen. The frozen solution was then lyophilized for 24 hours to remove water. A white powder could form containing zinc and amino acid when it is dried and form a neutral solution 15 (pH 7) when dissolved in water. Comparable formulations were formed with arginine and histidine but in the case of histidine the solution is neutralized with NaOH because the solution has a pH of 5.5.

### Example 12

Three middle aged female patients were treated with the composition described in Example 3 as a face cream. Each patient was instructed to apply the cream once or twice daily. Within three weeks, all showed improved skin condition including some wrinkle effacement and none experienced irritation or other adverse side effect.

Preferred embodiments of this invention are described herein, including the best mode known to the inventors for carrying out the invention. It should be understood that the illustrated embodiments are exemplary only and should not be taken as limiting the scope of the invention.

The foregoing description comprises illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings in the foregoing descriptions. Although specific terms may be employed herein, they are used only in generic and descriptive sense and not for purposes of limitation. Accordingly, the present invention is not limited to the specific embodiments illustrated herein.

What is claimed is:

1. A topical composition for treating skin wounds of an individual, comprising:
an extract including Zinc Gluconate, Lysine, Taurine, Potassium Gluconate, Carboxy Methyl Cellulose, Glycerin, Allantoin, water, Hydrogen chloride (HCl), Lidocaine, Niacin, Valine, L-Isoleucine, Proline, L-Arginine, Threonine, Glutamine, Histidine, Serine, Asparagine, Tyrosine, Cysteine, and pentapeptide including Enkephalin.

2. The composition of claim 1, wherein the Zinc Gluconate is present in an amount of about 5.0% by weight of said composition.

3. The composition of claim 1, wherein the Lysine is present in an amount of about 0.1% by weight of said composition.

4. The composition of claim 1, wherein the Taurine is present in an amount of about 1.0% by weight of said composition.

5. The composition of claim 1, wherein the Potassium Gluconate is present in an amount of about 1.0% by weight of said composition.

6. The composition of claim 1, wherein the Carboxy Methyl Cellulose is present in an amount of about 0.5% by weight of said composition.

7. The composition of claim 1, wherein the Glycerin is present in an amount of about 10% by weight of said composition.

8. The composition of claim 1, wherein the Allantoin is present in an amount of about 1.0% by weight of said composition.

9. The composition of claim 1, wherein the Lidocaine is present in an amount of about 3% by weight of said composition.

10. The composition of claim 1, wherein the composition maintains a pH balance ranges from about 6.5 to 7.5.

11. The composition of claim 1, wherein the composition is a solution.

12. The composition of claim 1, wherein the composition is provided in at least one form of a gel, a spray, liquid, lotion, and a cream.

13. The composition of claim 1, is applied to the affected skin of the individual for effectively treating skin wounds, relaxing muscle contractions, thereby reducing expression lines and skin wrinkles, and smoothening skin.

14. A method of treating skin wounds for an individual using a topical composition, comprising:
identifying an area of affected skin, which is affected by at least one of a wound, an expression line, and a skin wrinkle;
applying the topical composition to the affected skin;
wherein the topical composition comprising Zinc Gluconate, Lysine, Taurine, Potassium Gluconate, Carboxy Methyl Cellulose, Glycerin, Allantoin, water, Hydrogen chloride (HCl), Lidocaine, Niacin, Valine, L-Isoleucine, Proline, L-Arginine, Threonine, Glutamine, Histidine, Serine, Asparagine, Tyrosine, Cysteine, and pentapeptide including Enkephalin.

15. The method of claim 14, wherein the Zinc Gluconate is present in an amount of about 5.0% by weight of said composition, Lysine in an amount of about 0.1% by weight of said composition, Taurine in an amount of about 1.0% by weight of said composition, Potassium Gluconate in an amount of about 1.0% by weight of said composition, Carboxy Methyl Cellulose in an amount of about 0.5% by weight of said composition, Glycerin in an amount of about 10% by weight of said composition, Allantoin in an amount of about 1.0% by weight of said composition, and Lidocaine in an amount of about 3% by weight of said composition.

16. The method of claim 14, wherein the composition maintains a pH balance ranges from about 6.5 to 7.5.

17. The method of claim 14, wherein the composition is a solution.

18. The method of claim 14, wherein the composition is provided in at least any one form including a gel, a spray, liquid, lotion, and a cream.

19. A topical composition for treating skin wounds of an individual, comprising:
Zinc Gluconate in an amount of about 5.0% by weight of said composition, Lysine in an amount of about 0.1% by weight of said composition, Taurine in an amount of about 1.0% by weight of said composition, Potassium Gluconate in an amount of about 1.0% by weight of said composition, Carboxy Methyl Cellulose in an amount of about 0.5% by weight of said composition, Glycerin in an amount of about 10% by weight of said composition, Allantoin in an amount of about 1.0% by weight of said composition, and Lidocaine in an amount of about 3% by weight of said composition, water, Hydrogen chloride (HCl), and other potential additives includes, Niacin, Valine, L-Isoleucine, Proline, L-Arginine, Threonine, Glutamine, Histidine, Serine, Asparagine, Tyrosine, Cysteine, and pentapeptide including Enkephalin,
wherein the composition is effective in treating skin wounds, relaxing muscle contractions, thereby reducing expression lines and skin wrinkles, and smoothening skin.

20. The composition of claim 19, wherein the composition is provided in at least one form including a solution, a gel, a spray, liquid, lotion, and a cream.
